(19) United States
(12) Patent Application Publication
(10) Pub. No.: US 2024/0389864 A1
(43) Pub. Date: Nov. 28, 2024

(54) A SENSING ARRANGEMENT FOR OBTAINING DATA FROM A BODY PART USING ACCURATE REFERENCE VALUES

(71) Applicant: RTHS AB, Göteborg (SE)

(72) Inventors: Roy FORSLUND, Älvängen (SE); Thomas PETIG, Mölndal (SE); Fredrik MALMSTEN, Torslanda (SE); Lars CARLSSON, Torslanda (SE); Tommy HANSSON, Göteborg (SE)

(21) Appl. No.: 18/693,430

(22) PCT Filed: Sep. 20, 2022
(86) PCT No.: PCT/EP2022/076059

§ 371 Date: Mar. 19, 2024

(30) Foreign Application Priority Data
Sep. 23, 2021 (SE) 2130254-2

(51) Int. Cl. A61B 5/0205; A61B 5/024; A61B 5/117

(52) U.S. Cl. CPC A61B 5/0205; A61B 5/117; A61B 5/02416; A61B 2560/0242

(57) ABSTRACT

A sensing arrangement (100) for obtaining data from captured light reflected by, transmitted through, and/or modulated by a body part (110) of a person. The sensing arrangement comprises: illumination means (120) arranged to illuminate at least a portion of the body part (110); optical sensing means (130) arranged to capture light reflected by, transmitted through, and/or modulated by the body part (110); and a control unit (150) arranged to determine an identity of the person, determine one or more health parameters from the captured light, obtain, for at least one health parameter, a reference parameter associated with the person from a database, and determine a deviation of at least one health parameter from its corresponding reference parameter.

### TECHNICAL FIELD

The present disclosure relates to sensing arrangements for obtaining data from captured light reflected by, transmitted through, and/or modulated by a body part. The data may comprise biometric identification data and/or health parameters of a person. The sensing arrangements may be used in a control system for allowing ingress to a person.

### BACKGROUND

Considering recent pandemic and epidemic spreads of infectious diseases like SARS, MERS, and COVID-19, finding ways of limiting the spread is crucial. Since symptoms of disease may not be detected by the infected individual before he or she has become contagious, one viable option is to automatically scan vital signs, medical signs, or other related data of people entering locations where people move, meet, and gather, such as offices, airports, and arenas, and denying entry to people showing any signs of infectious disease and sickness. Automatic scanning, however, faces many challenges regarding accuracy, speed, and cost.

Data comprising various health parameters may be obtained from captured light reflected by and/or transmitted through a body part. Sensing arrangements for obtaining such data may also be used for obtaining biometric identification data. Checking the identity of a person entering the locations mentioned above can be a required compliment to checking health parameters.

There is a need for improved sensing arrangements.

### SUMMARY

It is an object of the present disclosure to provide improved sensing arrangements, which, i.a., offer improved performance by using more representative reference values.

This object is at least in part obtained a sensing arrangement for obtaining data from captured light reflected by, transmitted through, and/or modulated by a body part of a person. The sensing arrangement comprises: illumination means arranged to illuminate at least a portion of the body part; optical sensing means arranged to capture light reflected by and/or transmitted through the body part; and a control unit arranged to determine an identity of the person, determine one or more health parameters from the captured light, obtain, for at least one health parameter, a reference parameter associated with the person from a database, and determine a deviation of at least one health parameter from its corresponding reference parameter. The database is preferably a centralized or distributed database arranged separately from the sensing arrangement, i.e., physically distanced from the sensing arrangement and communicatively coupled to the sensing arrangement via wireless or wireline communication link. Thus, more than one sensing arrangement can use the same database in a cooperative manner. This way multiple sensing arrangements can be deployed at various respective physical locations, and a person can use any one of these sensing arrangements for health monitoring. Measurements can also be captured by other types of sensing arrangements, third party sensing arrangements, not specified here and not restricted to measuring the same health parameters as the sensing arrangement described here. Measurements from such third party sensing arrangements can be used to improve reference parameters used by the sensing arrangements described herein.

The disclosed sensing arrangement thus extracts data comprising various health parameters. A health parameter can be a vital sign, medical sign, or other related data. A health parameter can include presence and/or concentrations of molecules, antibodies, drugs, alcohol, proteins, cells, histopathological biomarkers etc. present in the blood flowing through the body part. Health parameters can, e.g., be captured from fluorescent light by endogenous or exogenous biomolecules.

It is often challenging to determine how large a deviation of health parameter can be before it is considered a problem. Therefore, a measured health parameter can be compared to a reference parameter, which represent a normal value, where normal can mean, e.g., deemed free from illness, disease, infection, medical conditions etc. The normal value, however, often varies significantly from person to person. The disclosed sensing arrangement therefore compares a measured health parameter of a person with a corresponding reference parameter of that particular person. This way, the deviation is more representative of, e.g., an illness, disease, infection, medical condition, etc., compared to existing methodologies.

According to aspects, the control unit is arranged to select one or more health parameters as respective conditional parameters, wherein each conditional parameter is mapped into a range in a predetermined set of ranges, and arranged to obtain, for at least one health parameter not selected as a conditional parameter, a reference parameter associated with respective mapped ranges of the one or more conditional parameters. This way, the one or more health parameters are compared to respective reference values that have been obtained under similar conditions as the current measurements. According to further aspects, the one or more health parameters comprise at least heart rate and blood pressure, and wherein at least the heart rate is selected as a conditional parameter.

According to aspects, the one or more health parameters, one or more reference parameters, and/or one or more conditional parameters comprise any of heart rate, blood pressure, blood oxygen saturation, breathing rate, body temperature, glucose level, and hemoglobin level. Analyzing the deviation of any of these parameters can provide indications of illness, disease, infection, medical condition etc.

According to aspects, the control unit is arranged to determine one or more environmental parameters associated with the environment of measurement conditions, wherein each environmental parameter is mapped into a range in a predetermined set of ranges, and arranged to obtain one or more reference parameters associated with respective mapped ranges of the one or more environmental parameters. This way, the one or more health parameters are compared to respective reference values that have been obtained under similar conditions as the current measurements. According to further aspects, the one or more environmental parameters comprise any of temperature, humidity, air oxygen concentration, elevation, date, location of the sensing arrangement, air pressure, and ambient lighting conditions.

According to aspects, the control unit is arranged to determine one or more individual parameters associated with the person, wherein each individual parameter is mapped into a range in a predetermined set of ranges, and arranged to obtain one or more reference parameters associated with respective mapped ranges of the one or more individual parameters. This way, the one or more health parameters are compared to respective reference values that have been obtained under similar conditions as the current measurements. According to further aspects, the one or more individual parameters comprise any of age, weight, and height.

According to aspects, measurements may be discarded or adjusted based on measurements captured by third-party sensing arrangements.

According to aspects, the reference parameters can be adjusted or discarded based on measurements captured by third-party sensing arrangements.

According to aspects, wherein at least one of the one or more health parameters is determined by analyzing a pulse plethysmograph signal, by analyzing fluorescent light emitted from endogenous molecules, and/or by analyzing exogenous activation. This way, a number of different health parameters can be obtained.

According to aspects, at least one of the one or more reference parameter is associated with a normal condition of the person where he/she is free from illness, disease, and/or infection. This normal condition may comprise a nominal value, which is then a scalar value from which a deviation can be determined. The normal condition may, however, also comprise a distribution, such as a statistical distribution indicative of a mean and a variance. This type of normal condition not only indicates what the expected value of a given heath parameter or set of health parameters should be, but also how much variation from a nominal value that can be expected in the person. By using more advanced reference parameters, such as parameters describing statistical distributions, more advanced statistical tests can be performed as part of determining the deviation. I.e., statistical hypotheses tests comprising deviation or no deviation from the reference distribution can be conducted.

According to aspects, at least one of the one or more reference parameter is associated with a previously known medical condition of the person.

According to aspects, the control unit is further arranged to upload at least one of the one or more health parameters to the database. According to further aspects, the reference parameter corresponding to the uploaded health parameter is updated based on the uploaded health parameter. Data from the body part may be obtained at a plurality of occasions. The data may, e.g., be captured every day over several months. Other intervals are also possible. Multiple measurements at multiple occasions makes it possible to establish and/or update individual reference parameter (base values) corresponding to respective health parameters. Establish and/or update individual reference parameter at a high frequency may provide much more representative values. This means that a plurality of sensing arrangements can establish accurate and personalized reference parameters in the database in a collaborative manner, which is an advantage since the reference parameters become more accurate this way.

According to aspects, the control unit is arranged to compare the deviation to a predetermined threshold value. This is a simple yet effective way of identifying, e.g., an indication of illness, disease, infection, medical condition etc., or other type of cause for concern.

According to aspects, the control unit is arranged to classify the deviation into a condition by computer-implemented classification model configured to classify the deviation. This can provide an accurate way of analyzing the deviation.

According to aspects, the identity of the person is determined from biometric identification data of the person obtained from the captured light. This is advantageous since no additional hardware is required.

According to aspects, the control unit is arranged to recommend an allowance or denial of admittance into an area past the sensing arrangement based on the deviation. This way, access can be provided only to people deemed free from illness, disease, infection, medical condition etc.

According to aspects, the control unit is arranged to recommend a follow up health test and/or consultation with medical expertise based on the deviation. This way, the cause of deviation can be investigated.

There are also disclosed herein methods associated with the same advantages as discussed above in connection to the different apparatuses. There is also disclosed herein computer programs, computer program products, and control units associated with the above-mentioned advantages.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated. Further features of, and advantages with, the present invention will become apparent when studying the appended claims and the following description. The skilled person realizes that different features of the present invention may be combined to create embodiments other than those described in the following, without departing from the scope of the present invention.

### BRIEF DESCRIPTION OF THE DRAWINGS

The present disclosure will now be described in more detail with reference to the appended drawings, where:

FIG. 1 illustrates an example sensing arrangement,

FIG. 2 is a plot showing a health parameter versus time,

FIG. 3 is a flow chart illustrating methods,

FIG. 4 schematically illustrates a control unit,

FIG. 5 shows a computer program product, and

FIGS. 6A-6C show different views of an example sensing arrangement.

### DETAILED DESCRIPTION

Aspects of the present disclosure will now be described more fully with reference to the accompanying drawings. The different devices and methods disclosed herein can, however, be realized in many different forms and should not be construed as being limited to the aspects set forth herein. Like numbers in the drawings refer to like elements throughout.

The terminology used herein is for describing aspects of the disclosure only and is not intended to limit the invention. As used herein, the singular forms “a”, “an” and “the” are intended to include the plural forms as well, unless the context clearly indicates otherwise.

The arrangements and methods disclosed herein can be used to measure health parameters using biosensors under common everyday conditions to a person. This conditions can be situations like, e.g., when a person arrives to work, is leaving work, is entering a movie theatre, arrives at a restaurant, etc. In general, the sensing arrangement 100 may be arranged in any location where people move, meet, and gather.

The sensing arrangement may be used in access control system for allowing access to a person deemed free from infectious disease and sickness and/or passing an authorization check. The access control system may comprise a gate or the like to temporarily block ingress. Alternatively, or in combination of, the access control system may comprise a light arranged to indicate if a person should be allowed access. The light can, e.g., switch between a green and red light to indicate if a person should be allowed access or not.

The disclosed sensing arrangement 100 can be used as a real time health scanner using automated scanning with no surface touch. The arrangement enables a safer way for people to meet, travel, and enjoy things together. The arrangement can e.g. detect symptoms of COVID-19 and other possible infections in just a few seconds or less. A symptom can be abnormal health parameters, e.g., deviating from some predetermined reference value.

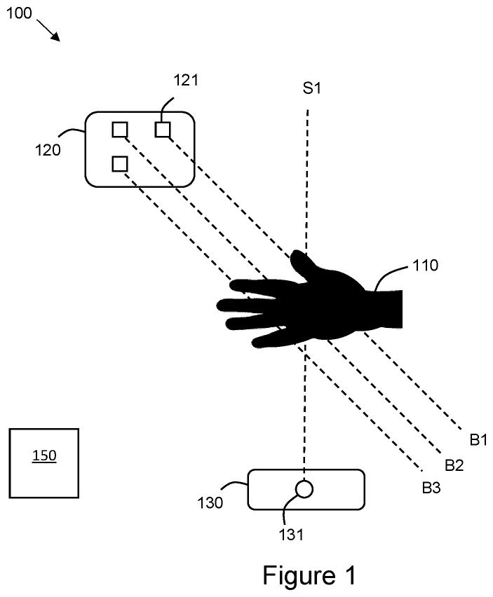

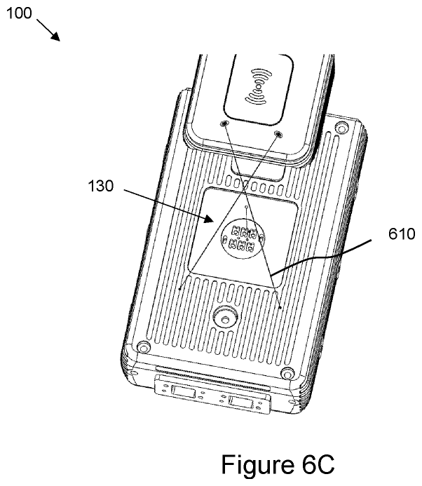

FIGS. 1 and 6A-6C show different embodiments of the disclosed sensing arrangement 100, which is suitable for obtaining data from an illuminated body part 110 of a person. The sensing arrangement comprises illumination means 120 arranged to illuminate at least a portion of the body part 110, and optical sensing means 130 arranged to capture light reflected by and/or transmitted through the body part 110. The optical sensing means may comprise one or more cameras.

The disclosed sensing arrangement extracts data comprising various health parameters. A health parameter can be a vital sign, medical sign, or related data. A health parameter can, e.g., be heart rate, blood pressure, blood oxygen saturation, breathing rate, body temperature, glucose level, hemoglobin level. A health parameter can also include presence and/or concentrations of molecules, antibodies, drugs, alcohol, proteins, cells, histopathological biomarkers etc. present in the blood flowing through the body part. Health parameters can be captured from fluorescent light by endogenous or exogenous biomolecules.

The health parameters can be obtained from the illuminated body part in various ways, e.g., by analyzing a pulse plethysmograph signal, by analyzing fluorescent light emitted from endogenous molecules (e.g., proteins), and/or by analyzing exogenous activation. These ways of obtaining health parameters rely on data in light that has in some way interacted with the body part. This includes light being passed through, being reflected, and/or being modulated by the body part. The fluorescent light may originate from autofluorescence and/or from a fluorescent marker administered (e.g., injected) in the blood flowing through the body part 110.

Existing measurement procedures for obtaining health parameters, like the ones already mentioned, require standardized procedures that most often require measurement conditions that are not natural or at least not frequent. Existing measurement procedures often require the person to be in a special location. In addition, specialized equipment is required in many cases. As an example, measuring blood pressure typically uses specialized equipment, such as a sphygmomanometer. In addition, blood pressure tests are typically performed at specialized locations and requires trained personnel. Furthermore, blood pressure tests typically require the person to rest for several minutes (e.g., 20 minutes) before the test is conducted. After that, the test itself can take several minutes. In general, it is often very important to rest beforehand to make sure that the measurement conditions are always the same. Otherwise, it is hard to compare the measurement to reference values and obtain meaningful data.

The disclosed sensing arrangement, on the other hand, can be arranged at convenient locations, such as the entrance to an office. It does not require trained personal, the measurements of the health parameters are quick, and the health parameters can be measured without requiring the person to rest beforehand. This opens up to possibility to monitor various health parameters of a person at a much higher frequency than what has been possible before. Due to all of the inconveniences of existing methods, the frequency of measuring, e.g., blood pressure, is often yearly or less often for an average person. With the disclosed sensing arrangement, health parameters can be measured daily or more often with minimal inconvenience. This frequency enables the detection of various health conditions at a much earlier stage compared to known methods.

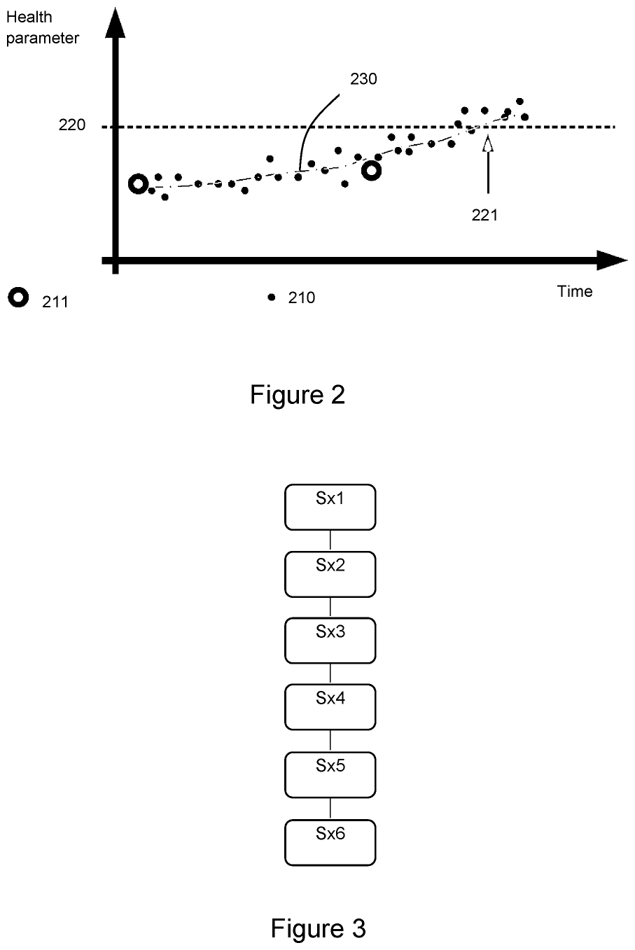

FIG. 2 shows measurements 210 of a health parameter using the disclosed sensing arrangement 100 for versus time, such as blood pressure versus time. In this particular example, the measurements 210 from the disclosed sensing arrangement 100 are accompanied by complementary measurements 211. The complementary measurements 211 are less frequent and may be obtained by other measurement equipment, such as specialized equipment located in a hospital or such. The complementary measurements 211 may be used to verify the accuracy of the measurements 210 from the disclosed sensing arrangement 100. A trend 230 of all measurements is increasing over time. At time instance 221, the trend passes a threshold 220. This threshold can indicate that the health parameter is within an undesired region, such as too high blood pressure according to some predetermined threshold. When passing the threshold, the sensing arrangement 100 may trigger an action, such as prompting a health checkup to investigate why the health parameter is in an undesired region.

It is often challenging to determine exactly where a threshold for an undesired value/region of a health parameter should be. Therefore, a measured health parameter can be compared to a reference parameter, which represent a normal value. It is often easier to determine a maximum allowable deviation from this reference value than to analyze absolute values. Here, normal can mean deemed free from illness, disease, infection etc. In other words, at least one reference parameter may be associated with a normal condition of the person where he/she is free from illness, disease, and/or infection. The normal value often varies significantly from person to person. For example, a normal body temperature is often 36.5-37° C. Measuring a body temperature of 37° C. may therefore be a normal value for one person, but may be an indication of illness for another person with a low normal body temperature. The disclosed sensing arrangement 100 therefore compares a measured health parameter of a person with a corresponding reference parameter of that particular person. This reference parameter is obtained from a database which is separated from the sensing arrangement, which means that several sensing arrangements can use the same database. The sensing arrangement may be communicatively coupled to the database using wireless and/or wireline communication links in a known manner, such as a Wi-Fi or a cellular connection to an access point which is then connected to the database via wireless connection, such as the Internet. A reference parameter may comprise a nominal value indicative of a normal value for a given person. The reference parameter may also comprise an expected deviation from this nominal value, which indicate how much a given health parameter is expected to deviate in a given person.

The reference parameter may also be associated with some average based on a group of people, in, e.g., society or in an office building. In that case, the reference parameter may be based on a weighted average of a personal value and an average value.

To summarize, there is disclosed herein a sensing arrangement 100 for obtaining data from captured light reflected by, transmitted through, and/or modulated by a body part 110 of a person. The sensing arrangement comprises: illumination means 120 arranged to illuminate at least a portion of the body part 110; optical sensing means 130 arranged to capture light reflected by and/or transmitted through the body part 110; and a control unit 150 arranged to determine an identity of the person, determine one or more health parameters from the captured light, obtain, for at least one health parameter, a reference parameter associated with the person from a database, and determine a deviation of at least one health parameter from its corresponding reference parameter.

The determination a deviation of at least one health parameter from its corresponding reference parameter may be comprised in a health test. The health test may also comprise triggering an action based on the deviation. For example, the control unit 150 may be arranged to recommend an allowance or denial of admittance into an area past the sensing arrangement 100 based on the deviation. Alternatively, or in combination of, the control unit 150 may be arranged to recommend a follow up health test and/or consultation with medical expertise based on the deviation.

According to aspects, the control unit 150 is arranged to compare the deviation to a predetermined threshold value. An example is a body temperature deviating more than 1° C. from the reference parameter. The magnitude of the deviation can be representative of various health conditions and is often more accurate than comparing a measured value of a health parameter to an absolute threshold value. The health test may comprise checking the magnitudes of a plurality of deviations. For example, only a deviating blood pressure may not be cause for concern, while both a deviating blood pressure and a deviating blood oxygen saturation may be a cause for concern. Furthermore, there may be several thresholds for each deviation or combinations of deviations, which, e.g., represent different levels of concern.

At least one of the one or more reference parameter may be associated with a previously known medical condition of the person. This way, an illness, e.g., may be detected on top of some other permanent or semi-permanent condition such as obesity.

According to other aspects, the control unit 150 is arranged to classify the deviation into a condition by computer-implemented classification model configured to classify the deviation. This is particularly advantageous when analyzing a plurality of health parameters. With five health parameters, e.g., there is a vast number of combinations of deviations that may be relevant to detect, and a vast number of combinations of deviations that are not relevant. Manually identifying combinations of thresholds for all combinations would be a very time consuming task. The classified condition may be binary, such as a cause for concern or not. However, the model may classify the deviation into a plurality of different classes, such as different degrees of concern.

The classification model may be a machine learning model, which generally relates to techniques where a model with a predetermined structure is adapted to give a desired function by providing some form of training. Any machine learning model may be trained with one or more test runs in a controlled environment and/or using simulations. For example, the computer-implemented classification model may be based on any of a neural network, a logistic regression algorithm, a random forest structure, and a K-nearest neighbors' algorithm, a support vector machine model, a Bayes algorithm, and a decision tree algorithm. These models are known in general and will therefore not be discussed in more detail herein.

It is appreciated that both the reference parameter per se and the processing of the deviation of the at least one determined health parameter from its corresponding reference parameter can be implemented using machine learning techniques. Thus, in case the system uses machine learning, then the reference parameter may in fact be embedded into the machine learning structure, which can be trained to recognize when the measured heath parameter deviates from the reference parameter. For instance, one or more sensing arrangements can be used to collect data about a given person. This data can then be annotated with companion data which indicates if the person is experiencing a health problem or not. A machine learning structure can then be trained using the collected data to detect when the sensed health parameter of a person no longer agrees with the expected health parameters, which could indicate that the person is unwell.

The identity of the person may be determined from biometric identification data of the person obtained from the captured light, which is advantageous since no additional hardware is required. Alternatively, or in combination of, the identity of the person may be determined from the RFID, near-field communication tags, identification papers/cards, keys, QR codes, passwords, or other identification means.

The sensing arrangement 100 may perform an authorization check to see if the person should be allowed ingress, e.g. Such authorized person can be a person that is allowed to enter, as in an office building. It can also mean anyone except people explicitly not allowed to enter, as in a passport control or an arena.

The biometric identification data may comprise any of vein pattern, artery pattern, and skin pattern. Other data extracted from the illuminated body part may also be used as biometric identification data. The biometric identification data may be used for an identification check, which comprises comparing obtained biometric identification data to a pre-determined set of biometric identification data values. The set of biometric identification data values can, e.g., comprise previously scanned vein patterns of a number of people, such as all employees in an office building.

Although identity cards, near-field communication tags, passwords etc. can be used for determining the identity of the person, their level of security is limited since they can be lost or stolen. Identification means using biometric data, on the other hand, can be intrinsically more secure. Biometric data commonly used for identification are fingerprints, iris scans, and facial recognition. Fingerprint scanning, however, often require physical contact between the finger and the scanner. This is unhygienic and is counterproductive to the prevention of contagion. This can be alleviated by using disinfectants, which may not be able to guarantee total disinfection and is an inconvenience and expense. Iris scanning is inconvenient and facial recognition may require extensive computational effort and computational training. Facial recognition may further feel invasive to the person.

As mentioned, the reference parameter should be representative of a normal condition to obtain accurate information from a deviation. In addition to obtaining a reference parameter associated with the person from a database, the reference parameter can be made more representative to a normal condition in a number of different ways. One particular example is to arrange the control unit 150 to select one or more health parameters as respective conditional parameters, wherein each conditional parameter is mapped into a range in a predetermined set of ranges, and obtain, for at least one health parameter not selected as a conditional parameter, a reference parameter associated with respective mapped ranges of the one or more conditional parameters. Any of the health parameters (blood pressure, blood oxygen saturation etc.) can be selected as a conditional parameter.

One particular example is that the one or more health parameters may comprise at least heart rate and blood pressure, where at least the heart rate is selected as a conditional parameter. In that case, if the measured heart rate is 105 bpm, the measured blood pressure can be compared to a reference parameter associated with, e.g., a heart rate span of 100-110 bpm. This way, the blood pressure is compared to a reference value obtained under similar conditions as the current measurements. As mentioned previously, existing measurement methodologies often rely on the person resting a long time before measurements are conducted. By using the disclosed sensor, however, measurements can be obtained when the person is out and about, e.g., on his/her way to work. The disclosed sensor is much faster since it does not require the rest time. Measurements can be much more frequent since the person can be tested at any time during the day and since the disclosed sensor can be arranged in a vast number of places.

Another example of making the reference parameter more representative to a normal condition is to arrange the control unit 150 to determine one or more environmental parameters associated with the environment of measurement conditions, wherein each environmental parameter is mapped into a range in a predetermined set of ranges, and arranged to obtain one or more reference parameters associated with respective mapped ranges of the one or more environmental parameters. The one or more environmental parameters may comprise any of temperature, humidity, air oxygen concentration, elevation, date, location of the sensing arrangement 100, air pressure, and ambient lighting conditions. This way, the one or more health parameters are compared to respective reference values that have been obtained under similar conditions as the current measurements. An environment parameter can be any information of the measurement conditions that may affect the representative value. This can include affecting the value itself (like, e.g., elevation) and/or affecting the accuracy of the measurement itself (like, e.g., a drift due to ambient lighting).

Another example of making the reference parameter more representative to a normal condition is to arrange the control unit 150 to determine one or more individual parameters associated with the person, wherein each individual parameter is mapped into a range in a predetermined set of ranges, and arranged to obtain one or more reference parameters associated with respective mapped ranges of the one or more individual parameters. The one or more individual parameters may comprise any of age, weight, and height. This way, the one or more health parameters are compared to respective reference values that have been obtained under similar conditions as the current measurements. An individual parameter can be any information of the person that may affect the representative value during a normal condition (e.g., free from illness). These can be medical or health information that changes relatively slowly compared health parameters that can indicate cause for concern (e.g., illness).

The control unit 150 may be arranged to upload at least one of the one or more health parameters to the database. In that case, the reference parameter corresponding to the uploaded health parameter is updated based on the uploaded health parameter.

According to aspects, data from the body part is obtained at a plurality of occasions. The data may, e.g., be captured every day over several months. Other intervals are also possible. Multiple measurements at multiple occasions can make it possible to establish and/or update individual reference parameter (base values) corresponding to respective health parameters. Establishing and/or updating individual reference parameter at a high frequency may provide much more representative values.

According to aspects, one or more reference values are established under one or more occasions. Preferably, the reference values are established from a plurality of occasions (e.g., more than five) to establish an accurate value. Preferably, reference values are also established for many different conditions, such as under different ranges of conditional, environmental, and/or individual parameters.

The sensing arrangement may obtain a plurality of health parameters. However, all of them are not necessarily obtained at every test occasion. Some health parameters may require a longer measurement time than desired. Therefore, it may be possible to only obtain such parameters occasionally (e.g., monthly, or weekly). In addition, some parameters may not be necessary to measure as often as other parameters to detect anomalies in the deviations. Which health parameters to be measured can be based on machine learning, based on previous measurements.

In some cases, the accuracy of an obtained health parameter can be increased by increasing the test time. Therefore, a health parameter may occasionally (e.g., monthly, or weekly) be obtained during a longer test time. Such selection can be based on machine learning, based on previous measurements.

The body part 110 preferably is a hand and/or a wrist. In that case, the illumination means may be arranged to illuminate the whole hand or a section of it, such as a 3 cm by 3 m square in the palm. The illumination means may be arranged to emit beams intersecting in a point on or in the body part. The body part may be other parts of a body, such as the earlobe, nose, foot etc. It may further be a large portion of a body, such as the torso with or without arms, or the whole body.

Different wavelengths penetrate human skin at different levels, which can reveal different data. Furthermore, a specific wavelength is absorbed by different amounts for different skin types. One factor affecting absorption for a wavelength is melanin concentration. Health parameters, such as blood oxygen saturation, can be obtained by analyzing light reflected and/or transmitted through the body part for two different discrete wavelengths. Other health parameters and other data can also be obtained by comparing captured light at two different discrete wavelengths. It is also possible to use three or more discrete wavelengths. According to aspects, the illumination means 120 of the sensing arrangement 100 is arranged to transmit a plurality of discrete wavelengths. In one aspect this means at least two, and in another aspect, this means at least three. According to further aspects, each of the discrete wavelengths are separated from each other by a distance in the spectrum, e.g., by 100 nm.

If one of the emitted discrete wavelengths is absorbed to a large amount, reflected, transmitted, and/or modulated light will be weak, i.e., have a low intensity. This results in a poor signal to noise ratio (SNR) for that the captured light of that particular wavelength. This means the ratio of the desired signal, i.e., the captured light, over undesired sources, such as ambient light from the sun or lighting arrangements. Undesired light can also be light from the illumination means 120 that has not been reflected and/or transmitted through the body part 110 since such light does not comprise any information of the body part. Such light can be emitted from a direct path between the illumination means 120 to the optical sensing means 130 or via a reflection from a wall or the like. If the body part is hand, a direct path can occur, e.g., between the fingers or at the edges of the hand. Preferably, the light illuminating the body part has passed through or has been reflected by blood. That way, various health parameters can be obtained accurately. As such, light only passing through the edge of a body part, without hitting blood, may also be undesired.

Using, e.g., three different wavelengths enables data to be obtained from a comparison of captured light of two different wavelengths even if the captured light at one wavelength has poor SNR. Here poor SNR means that the captured light does not yield data with sufficient accuracy. Using multiple wavelengths thus enables data acquisition with high accuracy for many different skin types. The inventors of the present disclosure have realized that six different wavelengths result in reliable data for a vast number of different skin types.

According to aspects, a health parameter is calculated using captured light at the two wavelengths presenting the best SNR out of all of the emitted wavelengths. However, light captured at one or more wavelengths with poor SNR are not necessarily ignored. These signals may be used to some extent when calculating health parameters, e.g., blood oxygen saturation. In that case, these signals may be used with some weighting to decrease their influence on the obtained health parameters.

Preferably, at least one discrete wavelength is within any of the visible spectrum, the infrared spectrum, and the ultraviolet spectrum. Even more preferably, at least one wavelength is within the blue/green spectrum and at least another wavelength is within the infrared spectrum. According to aspects, at least one wavelength is between 600-700 nm, preferably between 640-680 nm, and more preferably about 660 nm, and at least another wavelength is between 800-1000 nm, preferably between 850-890 nm, and more preferably about 870 nm.

Light comprising discrete wavelengths within the red/green spectrum is typically reflected at the surface of human skin. This reflection can therefore be used to reveal skin pattern, such as palm print, i.e. principal lines, secondary lines (wrinkles), and epidermal ridges. Infrared light on the other hand, tends to penetrate into the skin a few millimeters before it is reflected. Therefore, infrared reflections can be used to reveal blood vessel patterns, i.e. arteries, arterioles, capillaries, venules, and veins. Using different discrete wavelengths that penetrate the skin differently can therefore provide biometric data for different layers of the body part. Such biometric data can be used as unique identification means for a person.

According to aspects, the illumination means 120 comprises one or more emitters 121 and the illumination means is arranged to transmit at least three discrete wavelengths of light with respective transmit beams extending along respective extension directions B1-B3 from the one or more emitters to the body part 110. The optical sensing means 130 is in that case arranged at a distance from at least one emitter 121, is arranged facing the body part, and is arranged on a line S1 between the optical sensing means 130 and the body part 110. The line S1 is arranged at a non-zero angle with respect to an extension direction of at least one transmit beam B1-B3.

The optical sensing means may be arranged facing the body part and be arranged on a line S1 between the optical sensing means 130 and the body part 110. If the optical sensing means 130 comprises a single sensor (e.g., a camera) arranged in a housing, a normal direction of the housing is parallel to the line, i.e., the camera faces the body part, and the body part is arranged centrally in a picture captured by the camera. Normally, the optical sensing means 130 comprises a single housing, as is shown in FIG. 1. This housing may comprise a single sensor, as is shown in FIG. 1.

The optical sensing means 130 may be arranged at a distance from the illumination means emitters 120. This way, undesired leakage of light not reflected or passing through the body part can be reduced, which improves SNR. Preferably, the optical sensing means 130 is arranged at a distance from all emitters 121 of the illumination means 120. According to aspects, the distance between the optical sensing means 130 and the one or more emitters 121 is larger than the half the distance between the optical sensing means 130 and the body part. According to other aspects, the distance is at least 10 cm. According to further aspects, the distance is larger than a diameter of the illuminated area of the body part.

According to aspects, the optical sensing means is arranged in a separate housing from at least one emitter 121. However, the different housings may be connected to each other in various ways. A housing encapsulates at least a part of the optical sensing means. If the optical sensing means comprises a camera, the housing can be a camera housing. The housing can provide additional shielding from undesired light from the illumination means 120 from a direct path. Furthermore, undesired light that has been reflected can also be reduced, i.e., light from the illumination means that has not been reflected and/or transmitted through the body part but has been reflected on a wall or such. This way, SNR is improved further.

It is typically only relevant to capture light that has been reflected by or passed through the body part. Therefore, the optical sensing means may be arranged to capture light parallel with the line S1. This can be achieved with lenses, shielding, reflectors or the like. In other words, light not parallel to the line S1 is not captured, or at least such light is captured to a less extent compared to the parallel light. Here, to be parallel to the line S1 includes substantially parallel light as well, since the same technical effect is still obtained to some extent. According to aspects, parallel here means within plus-minus 20 degrees.

As mentioned, the line S1 is arranged at a non-zero angle with respect to an extension direction of at least one transmit beam B1-B3. In FIG. 1, the line S1 is arranged at a non-zero angle with respect to the respective extension directions of the transmit beams B1, B2, and B3. A non-zero angle with respect to a transmit beam means that the one or more sensors of the optical sensing means 130 are not arranged directly in the extension direction of that beam. This way, the undesired light emitted from a direct path between the illumination means 120 to the optical sensing means 130 is avoided.

When light hits the illuminated body part, a portion of the light is scattered. This scattered light is then reflected by and/or transmitted through the body part. Arranging the optical sensing arrangement 130 with the line S1 with a non-zero angle can therefore capture light comprising data of the body part.

Preferably, the non-zero angle is at least 10 degrees, and more preferably, the non-zero angle is at least 20 degrees, and even more preferably, the non-zero angle is at least 30 degrees. The non-zero angle may be a different angle with respect to different beams. Preferably, the line S1 is arranged at a non-zero angle with respect to the respective extension directions of all transmit beams B1-B3. However, if different sensors are used for respective wavelengths, it may be sufficient that each sensor is arranged at a non-zero angle with respect to the respective extension direction of the transmit beam of the corresponding wavelength.

The sensing arrangement may comprise illumination means 120 comprising one or more emitters arranged to illuminate at least a portion of the body part 110. An emitter is a light source arranged to transmit one or more beams, i.e., directed light. The illumination means 120 may comprise a respective emitter 121 for each wavelength. Alternatively, an emitter may emit a plurality or all discrete wavelengths. Multiple beams from one emitter may be overlapping. An emitter can comprise a plurality of light sources, such as LEDs, that can be grouped together. An emitter can be similar to an LED flashlight which comprises a plurality of LEDs and a reflector arranged to emit a beam.

If an emitter is arranged to transmit a plurality of different discrete wavelengths, it may comprise a plurality of interleaved light sources emitting different wavelengths.

Herein, to emit a single discrete wavelength means to emit light with a narrow bandwidth, such as the light from a light emitting diode (LED) or laser. Generally, the illumination means 120 herein comprise one or more emitters arranged to emit one or more beams of light, where each beam is associated with a beam width. For example, each such emitter may comprise a laser and/or an LED. A laser emits beam with monochromatic light, or rather light with a narrow linewidth, that is coherent. An LED emits light with a relatively low bandwidth, e.g., in the order 25 nm. An LED typically emits light as point source where a beam can be obtained using reflectors and/or lenses. Any light sources used in the emitter may utilize reflectors and/or lenses.

A beam typically has a peak intensity along an axis where the intensity drops symmetrically away from that axis. Arranging the optical sensing means 130 on a line S1 arranged at a non-zero angle with respect to an extension directions of at least one transmit beam B1-B3 therefore improves SNR. Preferably, the optical sensing means is arranged such that less than 10 percent of the light emitted by the illumination means 120 hits the optical sensing arrangement directly when no body part is arranged between the illumination means 120 and optical sensing means 130. Even more preferably, that amount light is less than 1 percent.

The illumination means 120 may alternatively, or in combination of, comprise a wideband emitter, such as an incandescent light bulb, in conjunction with one or more filters to emit one or more discrete wavelengths with narrow bandwidths, e.g., similar to an LED. One or more filters may also be used in conjunction with narrowband sources as well.

The optical sensing means 130 may comprise a respective sensor 131 for each wavelength. Each sensor may comprise a filter arranged for a single wavelength. The optical sensing means 130 may alternatively comprise a single sensor for a plurality or even all wavelengths. In that case, different filters may be arranged in front of the sensor at different times to capture light of different wavelengths. Capturing one wavelength at a time or using filters or timing enables the use of black and white cameras as a sensor(s). For example, if only a single discrete wavelength is transmitted at a time, there is no need to sort the light received by the camera sensor by wavelength in the camera sensor or the camera sensor output in order to extract the desired wavelength. Thus, a black and white camera may be used. An example resolution of the camera is 640 by 480 pixels. However, it is also possible to use other types of cameras and later distinguish different wavelengths using software or the like.

Having a respective sensor for each wavelength results in that all wavelengths can illuminate the body part simultaneously and all wavelengths can be captured simultaneously. This provides fast measurements and improved accuracy since all wavelengths comprise data of the same time instance. This significantly reduces problems introduced by movement of the body part. For example, it the data is analyzed by comparing absorption of two different wavelengths, it is preferable that the two different wavelengths are measured under as similar conditions as possible.

In case the optical sensing means comprises multiple sensors, the sensors are preferably spaced closely together so that they capture as similar images as possible. As mentioned, it may be preferable that the different wavelengths are measured under as similar conditions as possible.

In an example embodiment, the optical sensing means 130 comprises a camera arranged to capture an image at a rate of 600 Hz. Six discrete wavelengths are used, and the illumination means 120 is arranged to cycle through each of the six wavelengths at a rate of 600 Hz. Consequently, a picture of one of the wavelengths is captured at a rate of 100 Hz. Each wavelength is transmitted as a square pulse with a duration 0.28 milliseconds. Consequently, the illumination means is transmitting light at duty cycle of 25%, i.e. no light is emitted during 75% of the time, and each discrete wavelength has a duty cycle of 4.2%. Furthermore, the camera is triggered to capture an image during a pulse. This way, high illumination for each discrete wavelength can be obtained while keeping heating effects at a minimum. Other pulse durations and repetition rates are also possible. Constant illumination is also possible, one discrete wavelength at a time or all at once.

In another example embodiment, the illumination means 120 emits six different discrete wavelengths at the same time and the optical sensing means 130 comprises six different sensors arranged to capture respective wavelengths. Each sensor is a camera arranged to capture an image at a rate of 100 Hz. All of the wavelengths are transmitted as respective square pulse with a duration 0.625 milliseconds. Consequently, the illumination means is transmitting light at duty cycle of 25%, i.e. no light is emitted during 75% of the time. Furthermore, all cameras are triggered to capture an image during the pulses.

As is shown in FIG. 1, the extension directions of the transmit beams B1, B2, and B3 are parallel. More generally, the extension directions of a least two transmit beams B1-B3 may be parallel. In FIG. 1, the illumination means 120 comprises three different emitters 121 in a single housing. However, the illumination means may be distributed across different housings. In other words, the illumination means 120 may be distributed to transmit at least two beams from different locations. More specifically, the at least two emitters 121 are then arranged at a different physical location in relation to the body part compared to another emitter. This way, redundancy is introduced. If one emitter illuminates the body part such that little light is captured by the optical sensing means, another emitter at another location may provide light giving better data.

According to aspects, the sensing arrangement 100 comprises ranging means arranged to obtain a distance between the body part 110 and the optical sensing means 130. Knowing the distance between the optical sensing means and the body part is desired to be able to measure relative movements of the body part or sections of the body part, such as movement resulting from heart beats. The ranging means may comprise the optical sensing means 130 and a laser 140 arranged in proximity to the optical sensing means 130. It is beneficial to reuse the optical sensing means for this purpose since it saves costs and space. Furthermore, such arrangement can use constant illumination during the off time in an arrangement with illumination means 120 with a duty cycle. Arranging the laser in proximity to the optical sensing means can mean that they are in the same housing. According to aspects, the laser comprises a beam extending in a normal direction of a housing comprising the optical sensing means.

In an example embodiment, the laser 140 is parallel with the line S1, and the distance between the body part and the optical sensing arrangement is obtained from where the position of a dot of the laser on the body part is in the frame of a camera in the optical sensing arrangement. This way, complicated timing and triggering can be avoided. According to aspects, two or more lasers 140 are used for redundancy purposes. In some cases, the body part may have a shape where one of the ranging laser beams is reflected in an undesired dissection.

The ranging means may, however, also comprise any of a time of flight camera, a laser telemeter, an ultrasonic distance sensor, a radar sensor, or a stereo camera.

As mentioned, a health parameter can be blood oxygen saturation, which can be obtained from, e.g., magnitudes of at least two discrete wavelengths of the captured light. The magnitudes of at least two wavelengths in the reflected light, transmitted, and/or modulated light are then compared to an absorption spectrum of hemoglobin with and without oxygen to determine the blood oxygen saturation. In other words, the measured magnitudes are compared to expected magnitudes arising from a specific saturation of blood oxygen. Preferably, this measurement is averaged over a few seconds to capture accurate data. As mentioned, using captured light at the two wavelengths presenting the best SNR out of all of the emitted wavelengths may be used to obtain the blood oxygen saturation. Furthermore, wavelengths with different SNR can be assigned different weights to adjust their influence when obtaining the blood oxygen saturation. According two aspects, the illumination means arranged to transmit at least two discrete wavelengths of light with different absorption levels in blood.

Heart rate can be obtained from, e.g., variations over time of the magnitude of at least one discrete wavelength of the captured light. The heartbeat of a person causes many kinds of motion with a cyclical nature, such as the slight movement at the point on the wrist where pulse can be measured by touch. Such cyclical movement has a period commonly between 0.5 and 2 seconds. Relative movement of blood through a vein, e.g., can be indicated as amplitude variations of the reflected light at a single discrete wavelength. A plurality of wavelengths can also be averaged. Analyzing the cyclical nature of the amplitude variations can therefore provide the heart rate of the person. Preferably, this measurement is captured over a few seconds to capture accurate data. Captured light at the wavelengths presenting the best SNR out of all of the emitted wavelengths may be used to obtain the heart rate. Furthermore, wavelengths with different SNR can be assigned different weights to adjust their influence when obtaining the heart rate.

Breathing rate may be obtained from, e.g., variations over time of the magnitude of at least one discrete wavelength of the captured light. Similar to the heart rate, the breathing rate can be obtained by analyzing amplitude variations of the reflected light of one or more discrete wavelengths. The breathing rate can reveal itself by cyclical motion with a period commonly between 2 and 10 seconds. Using captured light at the wavelengths presenting the best SNR out of all of the emitted wavelengths may be used to the breathing rate. Furthermore, wavelengths with different SNR can be assigned different weights to adjust their influence when obtaining the breathing rate.

Blood pressure may be obtained from, e.g., a comparison of variations over time of the magnitude of at least one discrete wavelength of the captured light at different locations of the illuminated body part 110. Monitoring any type of variation over time for different parts of the body part allows for obtaining a time delay between a propagation of a movement, e.g. a pulse propagation of a blood arising from a heartbeat. This time delay can be used to analyze how much blood has been passed through a certain section during a certain time, which can give the blood pressure of the person. Using captured light at the wavelengths presenting the best SNR out of all of the emitted wavelengths may be used to the blood pressure. Furthermore, wavelengths with different SNR can be assigned different weights to adjust their influence when obtaining the blood pressure.

Any of body temperature, heart rate, breathing rate, blood oxygen saturation, and blood pressure can be used to determine if the person is alive. This adds another layer of security when biometric identification alone is not be secure enough. For example, fingerprints may be replicated or be detached, and it may be possible to mold a replication of a face.

According to aspects, the sensing arrangement 100 further comprises a thermal camera. In that case, the sensing arrangement may obtain body temperature. Furthermore, if the thermal camera is aimed at the face, the breathing rate can be obtained from measuring temperature differences of the face resulting from inhaled and/or exhaled air. The thermal camera is used to observe the colder and hotter skin areas, compared to the environment, preferably skin areas around the mouth and/or nose. This way of obtaining the breathing rate can be used as an alternative to or a combination with the way described above. A weighted average between the two different measurements may, e.g., be used. It is also possible to obtain the breathing rate from measurements of hot and cold air resulting from respiration and from measurements of relative movement of face. These two ways can be a compliment or alternative.

If the thermal camera is pointed at the face of the person, the blood pressure can be obtained using the optical sensing means 130 together with the thermal camera. In that case, the thermal camera is directed to body part different from the illuminated body part 110. The blood pressure is obtained from a measured time delay between an observation of a movement by the thermal camera and an observation of a movement by the optical sensing means 130. Both movements are movements of respective body parts resulting from the same heartbeat. Movement related to the heartbeat in the face can be observed by the thermal camera. Monitoring any type of variation over time in the signals received by the optical sensing arrangement can be correlated to the movement observed by the thermal camera. The time delay between the observations can then provide the blood pressure. This way of obtaining the blood pressure can be used as an alternative to or in combination with the way described above. A weighted average between the two different measurements may, e.g., be used.

The sensing arrangement 100 may comprise a display means arranged to show a live feed of the person and to show guiding means arranged guide to the person to an optimal distance between the person and the thermal camera. The guiding means can for example comprise a rectangle around the displayed face of the person that changes color to indicate distance. Green can indicate optimal distances and red can indicate incorrect distances. Optimal distances are distances allowing reliable sensor observations. The display means may comprise a monitor, like a television monitor, but it can also be a light strip or something similar.

The display means may alternatively, or in combination of, be arranged to show guiding means arranged to guide the person to an optimal distance between the illuminated body part of the person and the optical sensing means 130. The sensing arrangement 100 may comprise ranging means arranged to obtain a distance between the person and the thermal camera. This ranging means may comprises a time-of-flight camera arrangement. According to aspects, this ranging means may also comprise any of a laser telemeter, an ultrasonic distance sensor, a radar sensor, or a stereo camera.

According to aspects, the illumination means 120 comprises a reflector arranged to direct the light from the illumination means towards the illuminated body part. The reflector can facilitate equal illumination of the illuminated body part for the discrete wavelengths, from the point of view of the optical sensing arrangement. The number of emitters 121 and their placement can be optimized in conjunction with a reflector plate. The optimization tries to achieve evenly distributed illumination of the illuminated body part for the different wavelengths, from the point of view of the optical sensing means. There may be different amounts of diodes for one wavelength compared to another wavelength since the different diodes may have different intensities.

The sensing arrangement 100 may comprise a radar arranged to measure movement of a body part of the person. This movement measurement may replace other measurements using relative movements of the body or complement them, such as measurements obtaining a breathing rate of the person, obtaining heart rate of the person, and/or obtaining blood pressure of the person. The radar measurements can also be used in combination with other measurements.

According to aspects, at least one discrete wavelengths emitted by the illumination means 120 is arranged to excite a marker administered (e.g., injected) in the blood flowing through the body part 110. The marker, if binding to a specific molecular structure present in the blood, for example an antibody, will emit fluorescent light at a known and by the device detectable wavelength. Other structures can be alcohol, drugs, proteins, or molecules in general.

FIGS. 6A-6C show different views of an example sensing arrangement 100, where FIG. 6A shows an overview, FIG. 6B shows details of the illumination means 120, and FIG. 6C shows details of the sensing arrangement 130. An RFID arrangement 620 is arranged adjacent to the illumination means 120. This can be used as an alternative or complement to identification. In general, the RFID arrangement may be replaced by identification means using keys, QR codes etc.

In FIGS. 6A-6C, two guiding lasers with respective beams 610 are arranged adjacent to the illumination means. These beams intersect at point which has an optimal distance to the optical sensing arrangement 130. When the body part is placed between the optical sensing means 130 and the illumination means 120, these lasers will appear as a single dot on the body part when the body part is at an optimal distance from the optical sensing means 130, and as two dots for other distances.

FIGS. 6A-6C, the illumination means 120 comprises an emitter 121 with a plurality of sockets for LEDs. Different LEDs for a total of six different wavelengths are interleaved in this arrangement. The LEDs have viewing angles (related to beam width) of about 5-25 degrees for the various wavelengths. Note that other viewing angles are possible and that all wavelengths may have LEDs with the same viewing angle. All LEDs are aimed at the intersection point of the beams 610 of the guiding lasers.

In FIGS. 6A-6C, the optical sensing means 130 comprise six sensors 131 with respective filters for six different wavelengths. Each sensor is arranged in recess with roughly a rectangular shape. Respective beams of two ranging lasers 140 emerges from two locations between the two rows of sensors.

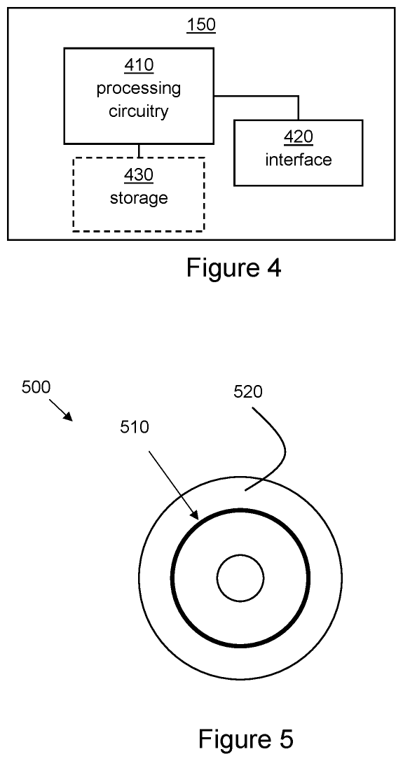

FIG. 3 is a flowchart illustrating methods. There is illustrated a method for obtaining data from captured light reflected by, transmitted through, and/or modulated by a body part 110 of a person. The method comprises
illuminating Sx1 at least a portion of the body part 110 with illumination means 120,
capturing Sx2 light reflected by and/or transmitted through the body part 110 with optical sensing means 130,
determining Sx3 an identity of the person,
determining Sx4 one or more health parameters from the captured light,
obtaining Sx5, for at least one health parameter, a reference parameter associated with the person from a database, and
determining Sx6 a deviation of at least one health parameter from its corresponding reference parameter.

Thus, the method describes aspects of the above disclosed techniques for obtaining data from an illuminated body part.

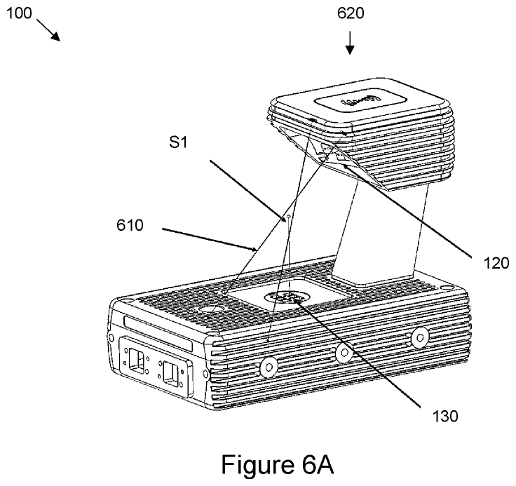

The control unit 150 may be arranged in the same housing as the illumination means 120 and/or optical sensing means 130. It may alternatively be in a housing connected to and/or directly adjacent to the illumination means 120 and/or optical sensing means 13. However, all, or part of, the control unit may be arranged separate from the illumination means 120 and/or optical sensing means 130. FIG. 4 schematically illustrates, in terms of a number of functional units, the general components of a control unit 150 of the sensing arrangement 100. Processing circuitry 410 is provided using any combination of one or more of a suitable central processing unit CPU, multiprocessor, microcontroller, digital signal processor DSP, etc., capable of executing software instructions stored in a computer program product, e.g. in the form of a storage medium 430. The processing circuitry 410 may further be provided as at least one application specific integrated circuit ASIC, or field programmable gate array FPGA.

Particularly, the processing circuitry 410 is configured to cause the sensing arrangement 100 to perform a set of operations, or steps, such as the methods discussed in connection to FIG. 3 and the discussions above, and also to set operating parameters of the system according to the discussions above. For example, the storage medium 430 may store the set of operations, and the processing circuitry 410 may be configured to retrieve the set of operations from the storage medium 430 to cause the device to perform the set of operations. The set of operations may be provided as a set of executable instructions. Thus, the processing circuitry 410 is thereby arranged to execute methods as herein disclosed.

The storage medium 430 may also comprise persistent storage, which, for example, can be any single one or combination of magnetic memory, optical memory, solid state memory or even remotely mounted memory. This storage medium may be configured to store one or more sets of configuration settings for the sensing arrangement 100.

The control unit 150 may further comprise an interface 420 for communications with at least one external device. As such the interface 420 may comprise one or more transmitters and receivers, comprising analogue and digital components and a suitable number of ports for wireline or wireless communication.

The processing circuitry 410 controls the general operation of the control unit 150, e.g., by sending data and control signals to the interface 420 and the storage medium 430, by receiving data and reports from the interface 420, and by retrieving data and instructions from the storage medium 430.

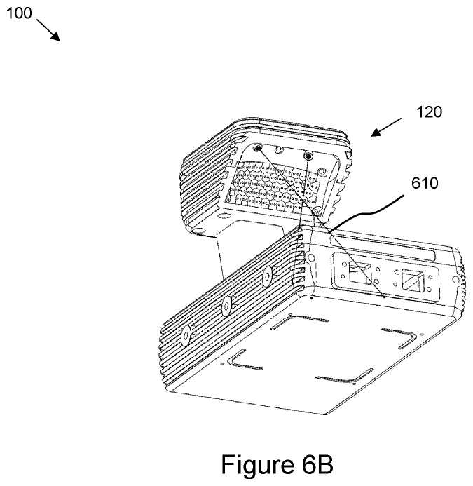

FIG. 5 illustrates a computer readable medium 510 carrying a computer program comprising program code means 520 for performing the methods illustrated in FIG. 3 and/or for executing the various functions discussed above, when said program product is run on a computer. The computer readable medium and the code means may together form a computer program product 500. This computer program product may comprise one or more sets of configurations for controlling the sensing arrangement 100 discussed above to perform the methods disclosed herein.

1. A sensing arrangement (100) for obtaining data from captured light reflected by, transmitted through, and/or modulated by a body part (110) of a person, the sensing arrangement comprising: illumination means (120) arranged to illuminate at least a portion of the body part (110); optical sensing means (130) arranged to capture light reflected by and/or transmitted through the body part (110); and a control unit (150) arranged to
determine an identity of the person,
determine one or more health parameters from the captured light,
obtain, for at least one health parameter, a reference parameter associated with the person from a database, and
determine a deviation of at least one health parameter from its corresponding reference parameter.

2. The sensing arrangement (100) according to claim 1, wherein the control unit (150) is arranged to
select one or more health parameters as respective conditional parameters, wherein each conditional parameter is mapped into a range in a predetermined set of ranges, and
obtain, for at least one health parameter not selected as a conditional parameter, a reference parameter associated with respective mapped ranges of the one or more conditional parameters.

3. The sensing arrangement (100) according to claim 2, wherein the one or more health parameters comprise at least heart rate and blood pressure, and wherein at least the heart rate is selected as a conditional parameter.

4. The sensing arrangement (100) according to claim 1, wherein the one or more health parameters, one or more reference parameters, and/or one or more conditional parameters comprise any of heart rate, blood pressure, blood oxygen saturation, breathing rate, body temperature, glucose level, and hemoglobin level.

5. The sensing arrangement (100) according to claim 1, wherein the control unit (150) is arranged to determine one or more environmental parameters associated with the environment of measurement conditions, wherein each environmental parameter is mapped into a range in a predetermined set of ranges, and arranged to obtain one or more reference parameters associated with respective mapped ranges of the one or more environmental parameters.

6. The sensing arrangement (100) according to claim 5, wherein the one or more environmental parameters comprise any of temperature, humidity, air oxygen concentration, elevation, date, location of the sensing arrangement (100).

7. The sensing arrangement (100) according to claim 1, wherein the control unit (150) is arranged to determine one or more individual parameters associated with the person, wherein each individual parameter is mapped into a range in a predetermined set of ranges and arranged to obtain one or more reference parameters associated with respective mapped ranges of the one or more individual parameters.

8. The sensing arrangement (100) according to claim 7, wherein the one or more individual parameters comprise any of age, weight, and height.

9. The sensing arrangement (100) according to claim 1, wherein at least one of the one or more health parameters is determined by analyzing a pulse plethysmograph signal, by analyzing fluorescent light emitted from endogenous molecules (proteins), and/or by analyzing exogenous activation.

10. The sensing arrangement (100) according to claim 1, wherein at least one of the one or more reference parameters is associated with a normal condition of the person where he/she is free from illness, disease, and/or infection.

11. The sensing arrangement (100) according to claim 1, wherein at least one of the one or more reference parameter is associated with a previously known medical condition of the person.

12. The sensing arrangement (100) according to claim 1, wherein the control unit (150) is further arranged to upload at least one of the one or more health parameters to the database.

13. The sensing arrangement (100) according to claim 12, wherein the reference parameter corresponding to the uploaded health parameter is updated based on the uploaded health parameter.

14. The sensing arrangement (100) according to claim 1, wherein the control unit (150) is arranged to compare the deviation to a predetermined threshold value.

15. The sensing arrangement (100) according to claim 1, wherein the control unit (150) is arranged to classify the deviation into a condition by computer-implemented classification model configured to classify the deviation.

16. The sensing arrangement (100) according to claim 1, wherein the identity of the person is determined from biometric identification data of the person obtained from the captured light.

17. The sensing arrangement (100) according to claim 1, wherein the control unit (150) is arranged to recommend an allowance or denial of admittance into an area past the sensing arrangement (100) based on the deviation.

18. The sensing arrangement (100) according to claim 1, wherein the control unit (150) is arranged to recommend a follow up health test and/or consultation with medical expertise based on the deviation.

19. A method for obtaining data from captured light reflected by and/or transmitted through a body part (110) of a person, the method comprising
illuminating (Sx1) at least a portion of the body part (110) with illumination means (120),
capturing (Sx2) light reflected by, transmitted through, and/or modulated by the body part (110) with optical sensing means (130),
determining (Sx3) an identity of the person,
determining (Sx4) one or more health parameters from the captured light,
obtaining (Sx5), for at least one health parameter, a reference parameter associated with the person from a database, and
determining (Sx6) a deviation of at least one health parameter from its corresponding reference parameter.
